(19) United States
(12) Patent Application Publication
(10) Pub. No.: US 2008/0308648 A1
(43) Pub. Date: Dec. 18, 2008

(54) Fragrance diffuser using multiple thin artificial wicks

(76) Inventor: Bradley Duane Pesu, Gilbert, AZ (US)

Correspondence Address: Bradley Duane Pesu, 3845 E. Edna Dr., Gilbert, AZ, 85296, US

(21) Appl. No.: 12/214,442

(22) Filed: Jun. 18, 2008

Related U.S. Application Data
(60) Provisional application No. 60/934,817, filed on Jun. 18, 2007.

(51) Int. Cl. A61L 9/04; B65D 85/00; B67D 5/00; B65D 83/00

(52) U.S. Cl. 239/44; 206/457; 222/3; 222/187

(57) ABSTRACT

Multiple artificial wicks can be used in water or solvent based fragrance mixtures that will fully empty the mixture from the container without the need to flip the wicks. The wicks can be made from strands of woven, twisted, braided, injection molded or extruded fibers that are natural or man-made. The length to width ratio of the wick should be a minimum of 30 for effective fragrance release. The wick gets fully saturated and remains saturated while providing continuous fragrance diffusion. There is no need to flip the wick over during use to refresh the fragrance diffusion. The wicks can also be fully dyed or pigmented with color. The wicks can also be partially dyed or pigmented with color. A ceramic bisque can also be placed above the fragrance oil mixture to provide a fragrance boost when in contact with the wick.

### CROSS REFERENCE TO RELATED APPLICATIONS

This application is based on provisional application Ser. No. 60/934,817, filed on Jun. 18, 2007, the entire disclosure of which is hereby incorporated herein by reference.

### BACKGROUND OF THE INVENTION

1. Field of the Invention

This invention relates generally to the field of a device which dispenses fragrance oil or other liquids. More specifically, this device uses a composition of multiple artificial wicks to volatilize fragrance oil to provide a consumer air care need for the home, office or other areas.

2. Background

Conventional reed fragrance diffusers use multiple thin reeds and claim that the oil is drawn up over 10″ along the whole internal length of the individual cellular sections of the reed. However, this is not true since you would need an internal diameter (“ID”) of 0.005″ to do this.

Most reeds have individual cellular section IDs much greater than this. Bamboo is also sold for use as a reed, but these do not even have cellular sections. In fact, the reed diffusers on the market diffuse mainly through absorption of the fragrance oil into the wood. Fragrance oil also coats the outside of the reed. Most reed fragrance diffuser retailers recommend flipping the reeds into the fragrance oil 2 or 3 times initially to wet the reeds. The retailers also recommend flipping them each week to refresh fragrance emission. Reed, bamboo and rattan are natural components derived from plants or trees. Some manufacturers have used a single, short, fat artificial wick with fragrance oil.

### BRIEF SUMMARY OF THE INVENTION

One aspect of the invention is that there is no need to flip artificial wicks unlike natural reeds which need to be flipped periodically in order for them to work. A natural reed is not porous enough to fully pull up the fragrance oil 10″ through capillary action. The reeds need to be flipped 2 or 3 times initially in fragrance oil in order to wet the outside of the reeds. This is messy and drops of fragrance oil can get on the consumer's hands and could cause surface damage to a table if drops of fragrance oil drip onto the surface. The reeds need to be flipped periodically in order to refresh the fragrance diffusion. Once again, the consumer's hands and table surface can come in contact with the fragrance oil causing irritation and/or damage. Reed diffusers don't work with small volume (1 fl oz) essential oils because there is not enough liquid to coat and absorb into the reed. Reed, bamboo and rattan are natural components derived from plants or trees.

Another aspect of this invention is that the minimum length/width of the artificial wick should be 30 for effective fragrance release. Single artificial wicks that are ½″ outer diameter (“OD”) and 3″ long have been sold as non-electric fragrance diffusers in prior art. The length to width ratio of these short wicks is around 6. The present disclosure uses multiple artificial wicks that are longer and thinner to increase surface area to increase fragrance emission. In fact, the surface area of a single wick that measures ½ OD by 3″ long is roughly half (4.71 sq in) that of two (2) wicks that measure ⅛″ OD by 12″ long at 9.42 sq in. In addition, wicks with a length to width ratio of 6 will not protrude outside the fragrance mixture container as much as a wick with a length to width ratio of 30. Thus, a wick with a length to width ratio of 6 decreases the effective surface area with the surrounding air, thus inhibiting fragrance release.

Another aspect of the invention is the wicks can be dyed any color for an aesthetic look which can match the fragrance oil mixture theme. You could combine orange and black wicks with a Halloween fragrance formula as an example.

Another aspect of the invention is that a color band can be put on the outside of the wick so that the wick will change color when a fragrance oil mixture is drawn up the wick. This would provide the consumer with a visual indication that the unit is working properly by showing a color change all the way to the top of the wick.

A further aspect of the invention is that the wick can release 2 grams/day or more of a volatile organic compound (VOC) compliant water or solvent based fragrance formula than conventional natural reed products.

Yet another aspect of the invention is that the wicks will totally empty the fragrance oil formula, unlike many reed diffusers which never empty during use. In order for the reed fragrance diffusion to be effective, expensive fragrance oils and/or carrier components are added which can make it hard for the unit to be volatile organic compound (VOC) compliant.

Another aspect of the invention is to use multiple wicks that are in contact with porous decorative ceramic bisque pieces to boost fragrance diffusion. Decorative ceramic bisque pieces can be placed in the bottleneck area so all multiple wicks touch and saturate the ceramic bisque. Testing has shown that this can increase fragrance diffusion by up to 50%. It should be noted that ceramic bisque pieces could also be used with reeds.

Other aspects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. <1> is a perspective view of the invention showing multiple artificial wicks inserted into a container holding a fragrance mixture.

FIG. <2> is a perspective view of the invention showing color bands on the outside of artificial wicks.

FIG. <3> is a perspective view showing multiple artificial wicks in contact with a porous ceramic bisque.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

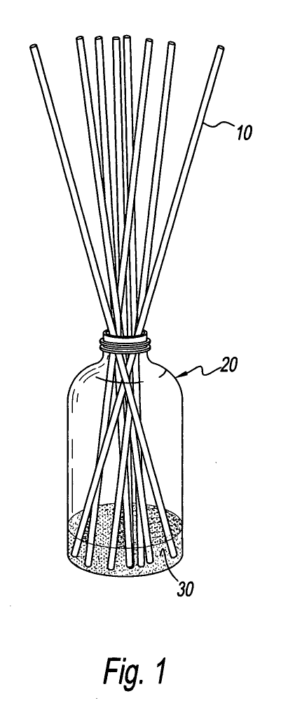

FIG. 1 shows several individual artificial wicks 10 inserted into a fragrance mixture container 20. The fragrance mixture container 20 holds a water or solvent based fragrance mixture 30. The artificial wicks can be made of strands of woven, twisted, braided or extruded fibers that are natural or man-made. By comparison, reeds are derived from plants or wood and are cut to length from their natural environment. Artificial wicks 10 can be made out of polymer fibers or even common natural materials like cotton through weaving, twisting, braiding or extrusion. The wicks are strong enough to support their own weight. The wicks color depends on the raw material used although normally they are white. The wick 10 can also be dyed or pigmented any color. As used herein, a dye or a pigment shall be called a colorant to indicate many ways of artificially coloring a wick. Testing has shown that multiple wicks that measure 2.5 mm OD and 12″ long perform well in water and solvent based fragrance mixtures 30.

Water based fragrance mixtures 30 can hold up to 10% fragrance oil. Emulsifiers are normally used to dilute the fragrance oil into the water. Care must be taken with the use of emulsifiers since high levels can clog the wick 10 during use. The fragrance mixture release rate for a water based solution can be over 20 grams/day using twelve wicks that measure 2.5 mm OD by 12″ long. It is desirable to release about 0.35 grams/day of fragrance oil over a month. Testing has been done on an 8 fl. oz liquid container volume at a 5% fragrance oil water based mixture using 4 wicks. This gives a total fragrance release rate of 0.4 grams/day.

Solvent based fragrance mixtures 30 can hold over 20% fragrance oil if a VOC compliant solvent is used. A typical VOC compliant solvent is Dowanol DPMA which has a vapor pressure of 0.08 mm HG at 21 deg C. The fragrance mixture release rate using 20% fragrance oil and 80% Dowanol DPMA using twelve wicks that measure 2.5 mm OD by 12″ long can be 2 grams/day. Testing in solvent based formulas using 30% fragrance oil and 70% DPMA with 12 wicks measuring 12″ long yields a total release rate averaging 2.0 grams/day. This means that 0.60 grams/day of fragrance is released. The solvent based formulas are volatile organic compound (VOC) compliant. By comparison, reed diffusers using solvent based formulas release about 2.5 grams of liquid/day initially using 8 reeds. The liquid release rate falls off significantly over time (1.0 gram/day) even if the reeds are flipped every week in a 10 fl.oz. bottle. Some never empty the liquid out of the bottle. At 15% fragrance oil concentration, the total fragrance oil release rate is 0.15 grams/day. By comparison, most electric plug in diffusers release about 0.3 grams/day of fragrance oil.

The fragrance mixture 30 could also be colored with a dye that would turn a white natural wick 10 the same color as the dye when the wick is put into the fragrance mixture.

The fragrance mixture container 20 can be opaque or translucent. Materials chosen for the fragrance mixture container should not degrade when in contact with the fragrance mixture 30. Materials could be glass, ceramic, metal, earthenware, porcelain, terracotta or even polymers like polypropylene, polyethylene and nylon.

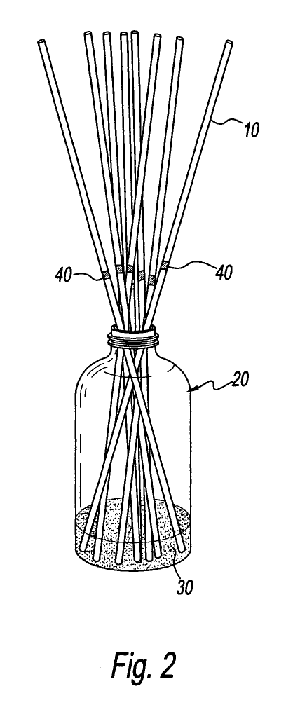

FIG. 2 shows several individual artificial wicks 10 with color bands 40 on the wick that are above the fragrance mixture 30. The color band can be either a pigment or dye and may comprise an ingredient that causes the color band to change color when in contact with a fragrance mixture. When this color band wick is inserted into the fragrance mixture 30, the mixture is drawn up the wick. The wick will change color above the location of the color band wick as the fragrance mixture is drawn up. Care must be taken to choose a water or solvent soluble dye or pigment for use with a water or solvent based fragrance mixture. The color band 40 can be applied to the wick 10 by printing a dye or pigment on the outside of the wick. A color band 40 that is ¼″ wide on the wick is normally sufficient to change the wick color above this location during use.

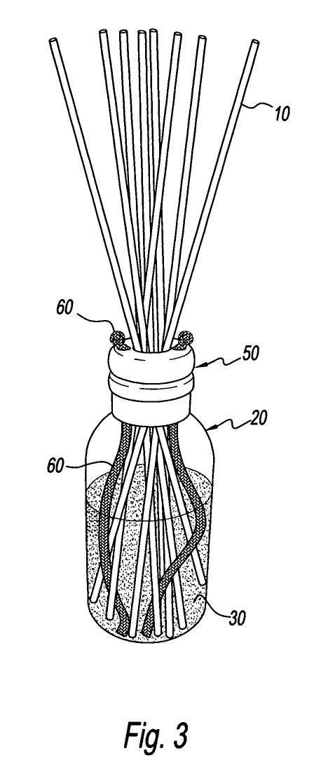

FIG. 3 shows a ceramic bisque 50 that is in contact with several artificial wicks 10. As the fragrance mixture 30 is drawn up the wicks, the wicks will saturate the ceramic bisque. The ceramic bisque is placed over the neck opening of the fragrance mixture container 20. This provides a way to increase the fragrance mixture release rate into the surrounding environment by using both the surface area of the ceramic bisque and wick. Wicks 60 that do not support their own weight could also be used with the ceramic bisque 50 by attaching them to the ceramic bisque, such as by tying a knot above the ceramic bisque or enlarging the wick 60 above an opening through the ceramic bisque. Note that the ceramic bisque could also be used with conventional reeds.

While the invention has been described in connection with a particular embodiment, it is not intended to limit the scope of the invention to the particular form set forth, but on the contrary, it is intended to cover such alternatives, modifications, and equivalents as may be included within the spirit and scope of the invention as defined by the appended claims.

1. A vapor dispensing device comprising: a liquid reservoir, multiple artificial wicks inserted into the reservoir and protruding from the reservoir, for vapor release from the wicks in the protruding area above the reservoir.

2. The device of claim 1, wherein the length to width ratio of the artificial wicks is a minimum of 30.

3. The device of claim 2, wherein the minimum number of artificial wicks is 2.

4. The device of claim 1, further comprising a water based fragrance composition in the reservoir.

5. The device of claim 1, wherein the liquid reservoir is a solvent based fragrance composition.

6. The device of claim 2 wherein the artificial wick comprises strands of woven, twisted, braided, injection molded or extruded fibers.

7. The device of claim 2 wherein all of the artificial wick is colored with a colorant.

8. The device of claim 2 wherein only a portion of the artificial wick is colored with a colorant.

9. The device of claim 1 further comprising a ceramic bisque in the area above the reservoir into which the multiple wicks protrude.

10. The device of claim 9 wherein the artificial wicks are in contact with the ceramic bisque.

11. A decorative fragrance dispenser comprising:
a liquid reservoir; at least two artificial wicks disposed within the reservoir and extending above the reservoir, each artificial wick having a width to length ratio of at least 30; a fragrance mixture disposed within the reservoir and in contact with the at least two artificial wicks.

12. The decorative fragrance dispenser of claim 11, further comprising a ceramic bisque disposed at an opening to the liquid reservoir and in contact with the at least two artificial wicks.

13. The decorative fragrance dispenser of claim 12, wherein at least one of the at least two artificial wicks is unable to support its own weight and is coupled to the ceramic bisque.

14. The decorative fragrance dispenser of claim 13, wherein the at least one wick coupled to the ceramic bisque extends through an opening in the ceramic bisque and comprises a knot or an enlarged portion above the opening larger than the opening.

15. The decorative fragrance dispenser of claim 11, wherein at least one of the at least two artificial wicks is colored with a colorant.

16. The decorative fragrance dispenser of claim 15, wherein the at least one colored artificial wick comprises a colorant band around a middle portion of the at least one colored artificial wick.

17. The decorative fragrance dispenser of claim 15, wherein the at least one colored artificial wick is colored for a majority of its length.
